(19) United States
(12) Patent Application Publication
(10) Pub. No.: US 2015/0066078 A1
(43) Pub. Date: Mar. 5, 2015

(54) Looped Tissue Fixation Device

(71) Applicant: Covidien LP, Mansfield, MA (US)

(72) Inventors: Daniel Broom, Branford, CT (US); Joshua Stopek, Minneapolis, MN (US); Timothy Sargeant, Guilford, CT (US); Nicholas Maiorino, Branford, CT (US); Arpan Desai, Hamden, CT (US); Saumya Banerjee, Hamden, CT (US); Timothy Flavin, Evanston, IL (US)

(21) Appl. No.: 14/389,887

(22) PCT Filed: Nov. 26, 2013
(86) PCT No.: PCT/US13/71795

§ 371 Date: Oct. 1, 2014

Related U.S. Application Data
(60) Provisional application No. 61/731,793, filed on Nov. 30, 2012.

(51) Int. Cl. A61B 17/06; A61B 17/04

(52) U.S. Cl. CPC A61B 17/06166; A61B 17/0401; A61B 2017/0406; A61B 2017/06176; A61B 2017/0412; A61B 2017/00876
USPC 606/228

(57) ABSTRACT

A tissue fixation device includes a barbed body in a general figure eight configuration including a first loop intersecting a second loop. The barbed body may include a third loop at a proximal end thereof, and/or a pledget at a distal end thereof. The configuration of the tissue fixation device may vary depending upon the performance requirements desired of the tissue fixation device for the envisaged application of use.

### CROSS-REFERENCE TO RELATED APPLICATIONS

This application claims the benefit of and priority to U.S. Provisional Patent Application No. 61/731,793, filed Nov. 30, 2012, the entire disclosure of which is incorporated by reference herein.

### BACKGROUND

1. Technical Field

The present disclosure relates to medical devices, and more particularly, to barbed surgical devices for tissue fixation.

2. Background of Related Art

Techniques for repairing damaged or diseased tissue are widespread in medicine. Wound closure devices, such as sutures and staples, as well as other repair devices like mesh or patch reinforcements, are frequently used for repair. For example, in the case of hernias, a surgical mesh or patch is commonly used to reinforce the abdominal wall. Typically, sutures, staples, and/or tacks are utilized to fix the surgical mesh or patch to surrounding tissue.

The current standard of care for laparoscopic ventral hernia repair, for example, involves the application of stay sutures that are placed both through a surgical mesh and trans-abdominally, and tied down just underneath the skin. Permanent sutures are typically used for this application. The sutures are placed around the perimeter of the mesh, and sometimes in the center to permanently tie the mesh to the area of herniation and to prevent the mesh from sliding within the peritoneum. For each stay suture, a surgeon will pierce the abdominal wall with a suture passer and grasp one end of a suture that has been pre-placed on the mesh, and pull the end of the suture through the abdominal wall and out past the skin. When the two ends of the suture are outside of the patient, the surgeon will pull up on the mesh and tie down a knot in the suture, compressing the abdominal wall and keeping the mesh tight against the peritoneum. The surgeon will then cut the excess suture and close the skin over the knot. This process may take about a minute or two for each stay suture, and may be associated with acute and/or chronic pain, likely due to compression of the abdominal wall and the nerves within it.

It would be advantageous to provide a fixation device that simplifies and shortens the time to secure a mesh, and limits or prevents pain caused by abdominal wall and nerve compression.

### SUMMARY

A tissue fixation device includes a barbed body in a general figure eight configuration including a first loop and a second loop. The barbs of the body may be single, compound, or combinations thereof. The barbed body may be a single continuous filament formed from biodegradable and/or non-biodegradable polymeric and/or metallic materials. The filament may be twisted, bonded, welded, fused, knotted, braided, entangled, crimpled, or otherwise joined at an intersection point between the first and second loops. Optionally, a third loop may intersect the first loop of the tissue fixation device. The third loop may be a separate looped filament that is free of barbs.

A pledget may be attached to the distal end of the barbed body. In embodiments, the pledget may be secured to a distal terminal end of the barbed body, while in some embodiments, the pledget may include a pair of openings through which the distal end of the barbed body may be laced through. The pledget may include a biocompatible coating. In embodiments, the coating is anti-adhesive, and in some embodiments, the coating may include surface reactive functional groups. In embodiments, the pledget may be magnetic. The pledget may also include barbs and/or darts to mechanically fix the pledget to tissue.

The proximal end of the body may include a cap. The cap may be magnetic or include a ring extending from a proximal end thereof to aid a clinician in placing the tissue fixation device within tissue. The proximal end may also include indicia.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a side view of a tissue fixation device in accordance with an embodiment of the present disclosure;

FIGS. 2A and 2B are side views of a single barb and a compound barb, respectively, of a filament of a tissue fixation device in accordance with embodiments of the present disclosure;

FIGS. 3A-3E are various views of embodiments of a tissue fixation device of the present disclosure including a pledget;

FIGS. 4A-4C are schematic illustrations of an exemplary method of using a tissue fixation device of the present disclosure; and

FIGS. 5A-5D are side views of embodiments of a tissue fixation device of the present disclosure including a cap.

### DETAILED DESCRIPTION

A tissue fixation device and method of using the same are described herein. While the present discussion and figures below depict exemplary embodiments of the present disclosure in terms of a tissue fixation device for use in hernia repair, the presently disclosed devices may be utilized in any surgical procedure requiring joining or positioning of tissue, or fastening of surgical implants thereto.

A tissue fixation device in accordance with the present disclosure includes a barbed body in a general figure eight configuration including a first loop and a second loop. The tissue fixation device described herein may be formed from any sterilizable biocompatible material that has suitable physical properties for the intended use of the device. The tissue fixation device may be fabricated from any biodegradable and/or non-biodegradable polymeric and/or metallic material that can be used in surgical procedures.

The term “biodegradable” as used herein is defined to include both bioabsorbable and bioresorbable materials. By biodegradable, it is meant that the material decomposes, or loses structural integrity under body conditions (e.g., enzymatic degradation or hydrolysis) or is broken down (physically or chemically) under physiologic conditions in the body such that the degradation products are excretable or absorbable by the body. Absorbable materials are absorbed by biological tissues and disappear in vivo at the end of a given period, which can vary, for example, from hours to several months, depending on the chemical nature of the material. It should be understood that such materials include natural, synthetic, bioabsorbable, and/or certain non-absorbable materials, as well as combinations thereof.

Representative natural biodegradable polymers include: polysaccharides such as alginate, dextran, chitin, chitosan, hyaluronic acid, cellulose, collagen, gelatin, fucans, glycosaminoglycans, and chemical derivatives thereof (substitutions and/or additions of chemical groups include, for example, alkyl, alkylene, amine, sulfate, hydroxylations, carboxylations, oxidations, and other modifications routinely made by those skilled in the art); catgut; silk; linen; cotton; and proteins such as albumin, casein, zein, silk, soybean protein, and copolymers and blends thereof; alone or in combination with synthetic polymers.

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt.

Representative synthetic biodegradable polymers include polyhydroxy acids prepared from lactone monomers such as glycolide, lactide, caprolactone, ε-caprolactone, valerolactone, and δ-valerolactone, carbonates (e.g., trimethylene carbonate, tetramethylene carbonate, and the like), dioxanones (e.g., 1,4-dioxanone and p-dioxanone), 1,dioxepanones (e.g., 1,4-dioxepan-2-one and 1,5-dioxepan-2-one), and combinations thereof. Polymers formed therefrom include: polylactides; poly(lactic acid); polyglycolides; poly(glycolic acid); poly(trimethylene carbonate); poly(dioxanone); poly(hydroxybutyric acid); poly(hydroxyvaleric acid); poly(lactide-co-(ε-caprolactone-)); poly(glycolide-co-(ε-caprolactone)); polycarbonates; poly(pseudo amino acids); poly(amino acids); poly(hydroxyalkanoate)s such as polyhydroxybutyrate, polyhydroxyvalerate, poly(3-hydroxybutyrate-co-3-hydroxyvalerate), polyhydroxyoctanoate, and polyhydroxyhexanoate; polyalkylene oxalates; polyoxaesters; polyanhydrides; polyester anyhydrides; polyortho esters; and copolymers, block copolymers, homopolymers, blends, and combinations thereof.

Some non-limiting examples of suitable non-degradable materials include: polyolefins such as polyethylene (including ultra high molecular weight polyethylene) and polypropylene including atactic, isotactic, syndiotactic, and blends thereof; polyethylene glycols; polyethylene oxides; polyisobutylene and ethylene-alpha olefin copolymers; fluorinated polyolefins such as fluoroethylenes, fluoropropylenes, fluoroPEGSs, and polytetrafluoroethylene; polyamides such as nylon, Nylon 6, Nylon 6,6, Nylon 6,10, Nylon 11, Nylon 12, and polycaprolactam; polyamines; polyimines; polyesters such as polyethylene terephthalate, polyethylene naphthalate, polytrimethylene terephthalate, and polybutylene terephthalate; polyethers; polybutester; polytetramethylene ether glycol; 1,4-butanediol; polyurethanes; acrylic polymers; methacrylics; vinyl halide polymers such as polyvinyl chloride; polyvinyl alcohols; polyvinyl ethers such as polyvinyl methyl ether; polyvinylidene halides such as polyvinylidene fluoride and polyvinylidene chloride; polychlorofluoroethylene; polyacrylonitrile; polyaryletherketones; polyvinyl ketones; polyvinyl aromatics such as polystyrene; polyvinyl esters such as polyvinyl acetate; etheylene-methyl methacrylate copolymers; acrylonitrile-styrene copolymers; ABS resins; ethylene-vinyl acetate copolymers; alkyd resins; polycarbonates; polyoxymethylenes; polyphosphazine; polyimides; epoxy resins; aramids; rayon; rayon-triacetate; spandex; silicones; and copolymers and combinations thereof.

In embodiments, the body or portions thereof of the tissue fixation device may be formed from polybutester, a copolymer of butylenes terephthalate and polytetramethylene ether glycol. For example, the tissue fixation device, or portions thereof, may be formed from the commercially available nonabsorbable polybutester monofilaments, sold under the trade name Novafil™ by Covidien. In some embodiment, the tissue fixation device, or portions thereof, may be formed from a copolymer of glycolic acid and trimethylene carbonate, such as, for example, Maxon™, commercially available from Covidien. In embodiments, the tissue fixation device, or portions thereof, may be formed from a terpolymer of glycolic acid, trimethylene carbonate, and dioxanone, such as, for example, Biosyn™, commercially available from Covidien.

In embodiments, the tissue fixation device may include: metals such as steel or titanium; metal alloys including degradable alloys such as iron-based or magnesium-based degradable alloys; and the like.

The body is formed from at least one filament that is fabricated from natural, synthetic, degradable, and/or non-degradable materials, as well as combinations thereof, as described above. The filament(s) may be formed using any technique within the purview of those skilled in the art such as, for example, extrusion, molding, casting, and/or spinning The filament(s) may also be drawn, oriented, annealed, calendared, crinkled, twisted, commingled, crimped, or air entangled to form the body.

The filament(s) of the body are barbed. The barbs may be single or compound barbs formed along a portion or the entire length of the body in specified or random patterns. Barbs may be formed from angled cuts in an outer surface of the body, or barbs may be molded on the outer surface of the body, such that an inner surface of the barb is positioned opposite to an outer surface of the body. The barbs may all be oriented in the same or different directions, and may be cut at the same or different barb angles. Compound barbs include an inner surface including at least two angled cuts disposed at first and second orientations, respectively, relative to a longitudinal axis of the body. Examples of compound barbs which may be utilized include those disclosed in U.S. Patent Application Publication No. 2009/0210006, entitled “Compound Barb Medical Device and Method”, the entire disclosure of which is incorporated by reference herein.

The surface area of the barbs may vary. For example, fuller-tipped barbs can be made of varying sizes designed for specific surgical applications. When joining fat and relatively soft tissues, large barbs may be desired, whereas smaller barbs may be more suitable for collagen-dense tissues. In some embodiments, a combination of large and small barbs on the same structure may be beneficial, for example, when used in a tissue repair with differing tissue layer structures. A combination of large and small barbs may be used within the same tissue fixation device such that the barb sizes are customized for each tissue layer to ensure maximum holding properties.

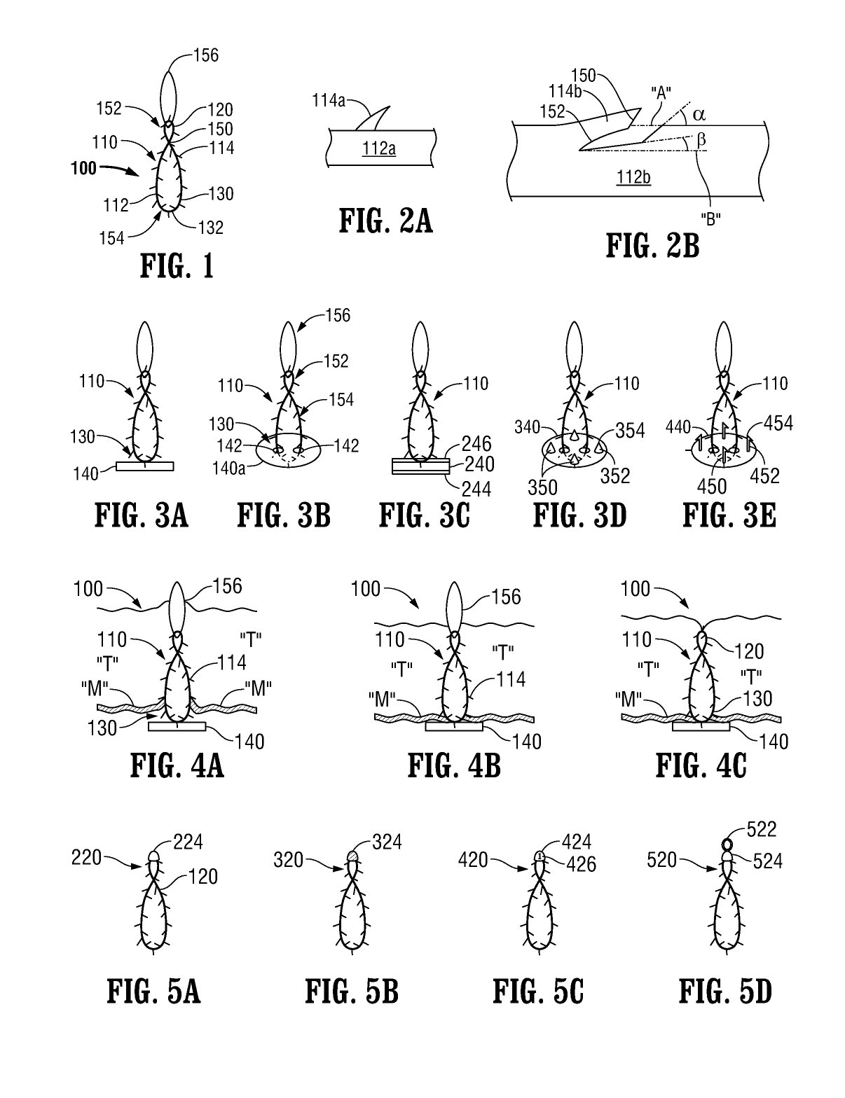

Referring now to the drawings, FIG. 1 illustrates a tissue fixation device 100 of the present disclosure. Tissue fixation device 100 includes a body portion 110 having a proximal end 120 and a distal end 130. The body portion 110 extends between the proximal end 120 and the distal end 130, and is illustrated as including a single continuous looped filament 112 twisted at an intersection point 150 to form a first loop 152 and a second loop 154. The filament 112 may be bonded, welded, fused, knotted, braided, entangled, or otherwise joined at intersection point 150. While the first and second loops 152 and 154 of the body portion 110 are illustrated as being different sizes, it is envisioned that the first and second loops may be the same size. Optionally, a third loop 156 may intersect the first loop 152 on the proximal end 120 of the body portion 110 of the tissue fixation device 100. The third loop 156 may be a separate looped filament that is free of barbs.

The barbs 114 on the filaments 112 of the body portion 110 may be single, compound, or a combination thereof. The barbs 114 may be disposed on any portion of the body portion 110 of the tissue fixation device 100. In embodiments, the barbs 114 may be disposed on at least the proximal half of the body portion 110. As illustrated, barbs 114 are disposed on the entire body portion 110, except the distal terminal end 132.

An exemplary single barb 114a on a filament 112a is illustrated in FIG. 2A, for example, and an exemplary compound barb 114b is illustrated in FIG. 2B. Compound barb 114b has an inner surface 150 including a first angle α, disposed at a first orientation relative to a longitudinal axis “A”' of filament 112b, and a second angle β having a second inner surface 152, disposed at a second orientation relative to a longitudinal axis “B” of filament 112b. The compound barb 114b may optionally include a third inner surface (not shown) disposed at a third orientation. In the embodiment shown, the first and second orientations are each disposed at different angles with respect to the longitudinal axis. In some embodiments, the barbs of the filaments may include a staggered arrangement of large or small barbs. In some embodiments, the filaments may have a random configuration of both large and small barbs. It will be understood that the barbs may include the same or different barb geometries.

The distal end of the tissue fixation device may be provided with a pledget, such as those shown in FIGS. 3A-3E. A pledget may be formed from plastic, polymeric, or other biocompatible materials, including non-degradable and/or degradable materials as described above. For example, in embodiments in which the distal end of the tissue fixation device is fabricated from a non-degradable material, a non-degradable pledget may be utilized, such as a pledget fabricated from a silicone or fluorocarbon based material, like polytetrafluoroethylene (e.g., TEFLON). Similarly, in some embodiments in which the distal end of the tissue fixation device is fabricated from a biodegradable material, a biodegradable pledget may be utilized, such as a pledget fabricated from a copolymer of lactide and glycolide.

In embodiments, a pledget may include magnetic material to aid a clinician in positioning the pledget against tissue with the use of an external magnet. A pledget may be fabricated from an absorbable and/or non-absorbable magnet material, such as a ferromagnetic metal. Suitable metals include iron ore (magnetite or lodestone), cobalt and nickel, rare earth metals like gadolinium and dysprosium, and alloys thereof. The pledget may also be made from composite materials such as ceramic or ferrite, alnico (a combination of aluminum, nickel and cobalt with iron), or triconal (a combination of titanium, cobalt, nickel and aluminum with iron). In some embodiments, a pledget may be formed from a polymeric material including ferromagnetic metal particles. The polymer may be any biodegradable and/or non-biodegradable polymer as described above. In embodiments, ferromagnetic metal particles may be freely admixed or co-extruded with the polymer forming the pledget, or may be tethered to the polymer through any suitable chemical bond. In some embodiments, the ferromagnetic metal particles may be spray or dip coated on a formed pledget.

Distal end 130 of the body portion 110 may be secured to the pledget 140 in a variety of ways. As illustrated in FIG. 3A, the distal end 130 of the body portion 110 may be secured to pledget 140 by chemically or thermally binding the pledget 140 thereto, such as by use of an adhesive, such as a hot melt adhesive, or by applying a binder, such as a powder, paste, or melt, and melting the binder to secure the distal end 130 to the pledget 140. In embodiments, the body and pledget may be secured mechanically. As illustrated in FIG. 3B, body portion 110 includes a pledget 140a including at least one pair of spaced opening 142 that are dimensioned to allow passage of the distal end 130 of the body portion 110 therethrough so that the pledget 140a may be secured to, and partially disposed within, the second loop 154.

In embodiments, a pledget may include a coating. The coating may be utilized to alter the physical properties on the surface of the pledget (e.g., enhance lubricity), or may provide a therapeutic benefit to tissue. In general, a coating may be applied to a surface of the pledget, or selective regions thereof, by, for example, spraying, dipping, brushing, vapor deposition, co-extrusion, capillary wicking, film casting, molding, etc.

Therapeutic agents include any substance or mixture of substances that have clinical use. Alternatively, a therapeutic agent could be any agent which provides a therapeutic or prophylactic effect; a compound that affects or participates in tissue growth, cell growth and/or cell differentiation; a compound that may be able to invoke or prevent a biological action such as an immune response; or a compound that could play any other role in one or more biological processes. A variety of therapeutic agents may be coated on a pledget, or incorporated into the tissue fixation device of the present disclosure. Moreover, any agent which may enhance tissue repair, limit the risk of sepsis, and modulate the mechanical properties of the tissue fixation device (e.g., the swelling rate in water, tensile strength, etc.) may be added to the material forming the tissue fixation device or may be coated thereon.

Examples of classes of therapeutic agents which may be utilized in accordance with the present disclosure include antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, and enzymes. It is also intended that combinations of therapeutic agents may be used.

Other therapeutic agents which may be in the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable therapeutic agents which may be included in the present disclosure include: viruses and cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, (α-IFN and γ-IFN)); erythropoietin; nucleases; tumor necrosis factor; colony stimulating factors (e.g., GCSF, GM-CSF, MCSF); insulin; anti-tumor agents and tumor suppressors; blood proteins; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); protein inhibitors; protein antagonists; protein agonists; nucleic acids such as antisense molecules, DNA, and RNA; oligonucleotides; and ribozymes.

As illustrated in FIG. 3C, a bottom surface 244 of a pledget 240 may include an anti-adhesive coating that acts as a barrier layer between the tissue fixation device and surrounding tissue to prevent the formation of adhesions, and a top surface 246 of the pledget 440 may be surface treated in order to promote adhesion to tissue. In embodiments, the top surface 246 may include a coating containing tissue reactive functional groups for fixation of the pledget 240 to tissue by crosslinking with reactive groups present in tissue such as primary amine groups, secondary amine groups, hydroxyl groups, carboxylic groups, sulfonic groups, combinations thereof, and the like. Such groups include compounds possessing chemistries having some affinity for tissue.

FIGS. 3D and 3E illustrate a pledget having mechanical means of attachment to tissue. The pledget may include mechanical barbs, grips, hooks, or darts to achieve, or enhance, adhesivity to tissue. As illustrated in FIG. 3D, a pledget 340 may include a plurality of mechanical darts 350, each including a base portion 352 tapering toward a sharp tip 354. The base portion 352 includes a larger diameter than the tip 354 for enhanced tissue fixation. As illustrated in FIG. 3E, pledge 440 may include mechanical darts 450, each dart 450 including an arm 452 having a sharp or pointed tip 454 for piercing and gripping tissue.

FIGS. 4A-4C illustrate an exemplary method for fixing a hernia mesh “M” to tissue “T” with a tissue fixation device of the present disclosure. As illustrated in FIG. 4A, tissue fixation device 100 is delivered to a surgical site and positioned with the third loop 156 extending through the tissue “T”, and the distal portion 130 abutting hernia mesh “M”. The tissue fixation device 100 may be delivered to the surgical site using a conventional suturing device, such as an Endo Close™ Single Use Suturing Device, commercially available from Covidien. For example, the tissue fixation device 100 may be hooked through a portion of a stylet of the Endo Close™ device and drawn within the cannula of the device. The Endo Close™ device may then be inserted through the tissue and released under the fascia without the need for a sharp piercing tip on the proximal portion of the tissue fixation device.

After the tissue fixation device 100 is delivered through the tissue “T”, a clinician may pull up on the third loop 156 such that the pledget 140 is compressed against the hernia mesh “M” and tissue “T”, as illustrated in FIG. 4B. The barbs 114 of the tissue fixation device 100 adhere to the hernia mesh “M” and/or tissue “T”, fixing the tissue fixation device 100 within the tissue “T”. The clinician may then cut the third loop 156 and remove it from the tissue fixation device 100, leaving the tissue fixation device 100 to hold the hernia mesh “M” against tissue “T”, as illustrated in FIG. 4C, without compressing the tissue “T” with a suture knot, as required by traditional devices. In embodiments, the proximal end 120 may be formed from a biodegradable material and the distal end 130 may be formed from a non-degradable material to aid in patient comfort by limiting the mass of the tissue fixation device within the tissue while retaining fixation integrity of the hernia mesh “M”.

The proximal end of the tissue fixation device may be capped. As illustrated in FIG. 5A, a proximal end 220 may terminate in a cap 224. The cap may be formed from any polymeric and/or metallic material as described above, to gather and crimp the filaments 112 together at a proximal terminal end thereof.

In embodiments, as illustrated in FIG. 5B, the cap 324 may be formed from a magnetic material, such as those described above in reference to the pledget.

In embodiments, the cap may include indicia, such as shapes, symbols, numerals, text, among other markings, for identifying the proximal end of the tissue fixation device. FIG. 5C illustrates a proximal end 420 including a cap 424 including indicia 426. The indicia may be in any shape and size to provide a visibly distinguishable mark or pattern on the proximal end of the tissue fixation device. In embodiments, indicia may be applied by utilizing ink that may be visualized under visible, infrared, ultraviolet, and/or by other wavelengths of light. In some embodiments, dyes may be utilized. Dyes include, but are not limited to, carbon black, bone black, FD&C Blue #1, FD&C Blue #2, FD&C Blue #3, FD&C Blue #6, D&C Green #6, D&C Violet #2, methylene blue, indocyanine green, other colored dyes, and combinations thereof. It is envisioned that visualization agents may also be used, such as fluorescent compounds (e.g., fluorescein or eosin), x-ray contrast agents (e.g., iodinated compounds), ultrasonic contrast agents, and MRI contrast agents (e.g., Gadolinium containing compounds). A variety of applicators within the purview of those skilled in the art may be used to apply the indicia, including, for example, syringes, droppers, markers or pen-like applicators, brushes, sponges, patches, combinations thereof, and the like.

FIG. 5D illustrates a proximal end 520 including a cap 524 having a ring 522 extending proximally therefrom. A ring 522 may be utilized to aid a clinician in pulling a tissue fixation device up through tissue.

Persons skilled in the art will understand that the devices and methods specifically described herein, and illustrated in the accompanying drawings, are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosed devices and methods based on the above-described embodiments. As such, further modifications and equivalents of the invention herein disclosed can occur to persons skilled in the art using no more than routine experimentation, and all such modifications and equivalents are believed to be within the spirit and scope of the disclosure as defined by the following claims.

What is claimed is:

1. A tissue fixation device comprising a barbed body in a general figure eight configuration including a first loop intersecting a second loop.

2. The tissue fixation device of claim 1, wherein the barbs are compound barbs.

3. The tissue fixation device of claim 1, wherein the body is a continuous filament twisted at an intersection point between the first and second loops.

4. The tissue fixation device of claim 1, wherein the body is fused at an intersection point between the first and second loops.

5. The tissue fixation device of claim 1, further comprising a third loop.

6. The tissue fixation device of claim 5, wherein the third loop is free of barbs.

7. The tissue fixation device of claim 1, further comprising a pledget disposed at a distal end of the body.

8. The tissue fixation device of claim 7, wherein the pledget includes a biocompatible coating.

9. The tissue fixation device of claim 8, wherein the coating is anti-adhesive.

10. The tissue fixation device of claim 7, wherein the pledget includes a proximal surface and a distal surface, the proximal surface including at least one dart extending proximally therefrom.

11. The tissue fixation device of claim 10, wherein the dart terminates in a sharp tip.

12. The tissue fixation device of claim 7, wherein the pledget includes surface reactive functional groups.

13. The tissue fixation device of claim 7, wherein the pledget is magnetic.

14. The tissue fixation device of claim 7, wherein the pledget is secured to a distal terminal end of the body.

15. The tissue fixation device of claim 7, wherein the pledget includes at least one pair of spaced openings, and wherein at least one of the first and second loops is laced through the openings.

16. The tissue fixation device of claim 1, wherein a proximal end of the body includes a cap.

17. The tissue fixation device of claim 16, wherein the cap is magnetic.

18. The tissue fixation device of claim 17, wherein the cap is ferromagnetic.

19. The tissue fixation device of claim 16, wherein the cap includes indicia.

20. The tissue fixation device of claim 16, wherein a ring extends from a proximal end of the cap.

21. A tissue fixation device comprising:
a barbed body defining a longitudinal axis and including a proximal end and a distal end, the barbed body including at least two barbed filaments intersecting at at least three common points along the longitudinal axis to form at least two integral loops, the common points including a proximal point at the proximal end, a distal point at the distal end, and at least one point between the proximal and distal ends.

22. The tissue fixation device of claim 21, further comprising a separate looped filament intersecting the loop at the proximal end of the body.
